(19) United States
(12) Patent Application Publication
(10) Pub. No.: US 2026/0151353 A1
(43) Pub. Date: Jun. 4, 2026

(54) Device and Methods For Improving the Efficacy and Interpersonal Safety of 1.62% Testosterone Gel

(71) Applicant: Stephen Bennis Gerken, Milwaukie, OR (US)

(72) Inventor: Stephen Bennis Gerken, Milwaukie, OR (US)

(21) Appl. No.: 18/964,598

(22) Filed: Dec. 1, 2024

(51) Int. Cl. A61K 9/70; A61K 9/06; A61K 31/568; A61K 47/10; A61K 47/14; A61K 47/32

(52) U.S. Cl. CPC A61K 9/7084; A61K 9/06; A61K 31/568; A61K 47/10; A61K 47/14; A61K 47/32

(57) ABSTRACT

A transdermal testosterone delivery device and methods are disclosed, which improve on the delivery efficiency, therapeutic efficacy, and mitigation of certain risks with respect to 1.62% testosterone gel. The device consists of metalized foil, adhesive membrane, gauze, and paper. The methods include a method of modifying 1.62% testosterone gel to create an ointment which may be administered without material adverse consequence, a method of sizing the device to the magnitude of the applied dose, and a method of timing the application and duration of doses. The device and methods provide therapeutically effective treatment of testosterone deficiency with a better subjective experience of quality of life, while using less testosterone, compared to 1.62% testosterone gel used in isolation according to its labeling.

### REFERENCES

U.S. Pat. No. 4,704,282A
U.S. Pat. No. 5,152,997A
Patent US 20200330397 A1
1.62% Testosterone Gel [package insert]. Glasgow, Kentucky: Amneal Pharmaceuticals LLC, 05-2019.
1.62% Testosterone Gel [package insert]. Baltimore, Maryland: Lupin Pharmaceuticals, Inc; February 2022.
Lewis T, Goldstein I. Transfer of topical testosterone preparations to children or spouses. J Sex Med. 2009 October; 6(10):2649-52. doi: 10.1111/j.1743-6109.2009.01478.x. PMID: 19817980.
Miller M G, Rogol A D, Zumbrunnen T L. Secondary exposure to testosterone from patients receiving replacement therapy with transdermal testosterone gels. Curr Med Res Opin. 2012 February; 28(2):267-9. doi: 10.1185/03007995.2011.652255. Epub 2012 Jan. 20. PMID: 22185432.
Meite, Ladji & Soro, Baba & Narcisse, Aboua & Mambo, Veronique & Sory, Traore & Mazellier, Patrick & de Iaat, Joseph. (2016). Qualitative Determination of Photodegradation Products of Progesterone and Testosterone in Aqueous Solution. American Journal of Analytical Chemistry. 07. 22-33. 10.4236/ajac.2016.71003.

### BACKGROUND OF THE INVENTION

This invention is in the field of transdermal drug delivery. More specifically, it pertains to a device and methods for improving the transdermal delivery of testosterone across unbroken nonscrotal skin. The device and methods improve the therapeutic effectiveness for the person using the device and methods, and also improve the mitigation of the risk of accidental transfer of testosterone to others.

Androgen deficiency in men results from primary or secondary hypogonadism, congenital or acquired, caused by one of several specific conditions. Specific underlying conditions may include cryptorchidism, bilateral torsion, chemotherapy, removal or congenital absence of testicles, Klinefelter's syndrome, toxic damage from alcohol or heavy metals, radiation damage, tumors, or trauma.

Androgen deficiency status is measured via a laboratory test of blood serum testosterone concentration. Widely accepted values of the normal range for adult men are from 300 to 1,050 ng/dl. Values below 300 ng/dl are strong evidence of androgen deficiency for men of all post-pubertal ages.

Androgen deficiency in men is associated with multiple adverse medical conditions, including but not limited to decreased libido, erectile dysfunction, fatigue, depression, low bone mineral density including early-onset osteoporosis, decreased muscle mass, and metabolic syndrome. Treatment of androgen deficiency is via supplementation with exogenous testosterone, also called hormone replacement therapy (HRT) or testosterone replacement therapy (TRT). TRT can also used as a component of gender-affirming treatment.

Oral administration of testosterone is not widely considered a viable method of supplementation due to rapid degradation of testosterone by the liver. Viable supplementation routes include oral administration of testosterone undecanoate, intramuscular injection of testosterone esters, subcutaneous injection of testosterone enanthate (XYOSTED®), implantation of subcutaneous testosterone pellets, five-layer transdermal patches (AndroDerm®), and transdermal gel (AndroGel® and generic 1.62% testosterone gel). Generic 1.62% testosterone gel consists of ethanol (72.22% volume/volume in Amneal®, 68.1% mass/mass in Lupin®), carbopol 980 (Amneal®) or carbomer homopolymer type C (Lupin®), isopropyl myristate, purified water, sodium hydroxide, and testosterone 1.62% mass/mass.

Prior art in the field of transdermal testosterone transfer, including but not limited to U.S. Pat. No. 5,152,997, documents that when testosterone is dissolved in an ethanol-based carrier and applied to non-scrotal skin, the transdermal flux of testosterone across the skin is greatest when the testosterone is at a subsaturation concentration, with maximal 24 hr cumulative flux achieved at concentrations in the range of ten to fifteen mg testosterone per ml of carrier, and near-maximal 24 hr cumulative flux in the range of five to 20 mg testosterone per ml of carrier.

1.62% testosterone gel is within this general band of subsaturation effectiveness when dispensed. With 1.62% testosterone gel, the 1.62% value is a mass:mass ratio of 20.25 mg testosterone per 1.25 g dispensed gel. The 1.62% value is not a mass:volume ratio as used in the referenced patent. Labeling of 1.62% testosterone gel does not indicate a gel density, nor a volume per dispensed dose. Field measurements indicate the density of 1.62% testosterone gel as dispensed is approximately 0.84 g/ml, at which density the mass:volume concentration of testosterone in gel would be approximately 13.6 mg/ml. This concentration is within the range of greatest 24 hr cumulative flux as observed in the U.S. Pat. No. 5,152,997 patent.

However, when applied per labeling, the gel is spread into a thin film with very high surface area for its volume. In conditions conducive to the patient remaining unclothed for the time period specified in labeling, the alcohol of the gel rapidly evaporates into surrounding air and disperses. As the alcohol evaporates, the concentration of nonabsorbed testosterone in nonevaporated gel may be expected to rise, which would bring the testosterone concentration out of the high-flux concentration range. Per the findings in the prior art, this change in concentration would be expected to reduce the total flux of testosterone across the skin, as compared to the flux occurring at the moment of application and prior to evaporation.

Due to the continuing presence on the skin of the nonevaporated gel components and the nonabsorbed testosterone, transdermal 1.62% testosterone gel has a known adverse side effect that when administered per labeling, testosterone can be transferred from the skin of the patient to animals or people with whom the patient has close physical contact. This transfer of testosterone via skin contact has been observed to cause undesired virilization of people other than the patient (see e.g. Lewis and Goldstein, 2009 or Miller, Rogol, and ZumBrunnen, 2012). Per FDA labeling, the approved mitigation of the risk of accidental testosterone transfer is for the patient to clothe the entire torso and upper arms at all times, and/or to remove the transdermal testosterone gel by washing with soap and water prior to anticipated interpersonal contact.

In actual practice, the approved mitigation of the risk of accidental transfer of testosterone contributes to the physical isolation of the patient. Additionally, the perception of residual risk in the presence of mitigation further contributes to the physical isolation of the patient. In plain terms, others decline to embrace the patient, or limit the length and/or contact area of embrace. Also, the responsible patient having first-hand experience of the virilizing effects of testosterone self-inhibits from pursuing such contact. Other intimate contact involving touch on or with the bare arms, collarbone, shoulders, neck, and other areas of the torso are also not initiated by others, and/or not pursued by the responsible patient. These factors lead to patient loneliness, lack of physical contact, and material reduction of intimate contact while medicated, compared to medication methods not associated with the risk of interpersonal medication transfer. Further, removal of medication in anticipation of interpersonal physical contact leads to patient fatigue, lack of libido, inability to achieve or sustain erection, depression, and other physical and psychological symptoms associated with androgen deficiency. These consequences of removal of medication also contribute to material reduction in physical contact and intimate contact, compared to medication methods not associated with the risk of interpersonal medication transfer.

The research subject in the development of the invention has a medically documented history of androgen deficiency. His androgen deficiency was successfully treated with AndroDerm® for many years, most recently at a dose of AndroDerm® 4 mg twice daily. At this dose, the patient applied one AndroDerm® 4 mg patch circa 8 am-9 am daily, and applied a second AndroDerm® 4 mg patch circa 5 pm-6 pm daily, removing both patches in the evening of the following day. Each AndroDerm® 4 mg patch contained 19.5 mg of testosterone. The “4 mg” in the product name AndroDerm® 4 mg referred to the nominal or expected amount of the 19.5 mg of testosterone present in the AndroDerm® which crossed the skin and entered peripheral circulation. At this dosing, the patient thus applied 39 mg of testosterone daily as the sum of the testosterone in the morning done and the testosterone in the afternoon dose.

Following the commercial discontinuation of AndroDerm®, the research subject used Amneal® 1.62% testosterone gel per its labeled instructions at a dosage of four pumps daily, which is the maximum on-label dose of 1.62% testosterone gel and corresponds to a nominal daily testosterone application of 81 mg. During the initial adjustment period, the research subject documented a number of significant deficiencies in the therapeutic effectiveness of 1.62% testosterone gel as compared to his prior experience with AndroDerm®, including worse mood control, worse impulse control, worse focus, worse work productivity in core tasks requiring sustained focused concentration, worse energy availability throughout the day, significant reduction in interpersonal physical contact generally, significant reduction in many forms of intimate contact with his intimate partner, and worse stamina in aerobic physical activity. The research subject increased caffeine intake threefold compared to personal baseline in an attempt to address deficiencies in focus, sustained concentration, and energy availability; this increase in caffeine consumption was ineffective. The research subject materially curtailed routine life activities, such as driving an automobile late in the afternoon or in the evening, due solely to the therapeutic ineffectiveness of 1.62% testosterone gel as compared to his prior experience with AndroDerm®.

After approximately 17 days of 1.62% testosterone gel therapy, a pre-dose morning testosterone serum concentration was performed on the research subject, for purposes of dose titration and/or evaluation of efficacy of therapy. The result of this test was a testosterone level of 104 ng/dl. This value is well below the low end of the normal range and reinforces analytically the experiential observations that the 1.62% testosterone gel used per its labeling did not address the research subject's androgen deficiency. A better therapy was sought.

### SUMMARY OF THE INVENTION

The invention comprises a device, an ointment, and related methods. The device comprises gauze, paper, adhesive membrane, and metalized foil. The ointment comprises 1.62% testosterone gel and glycerin anhydrous USP 99.5% in a volume:volume ratio of between 6:1 and 4:1. The methods comprise a method of combining 1.62% testosterone gel with glycerin anhydrous USP 99.5% to create the ointment, a method of constructing the device, and a method of usage of the ointment with the device in order to obtain clinically appropriate testosterone blood levels while mitigating the risk of interpersonal testosterone contamination.

Two different recipes of the ointment have been successful at producing stable testosterone levels within the normal range without excessive adverse effect.

In Recipe A, the ointment comprises a mixture of 1.62% testosterone gel with glycerin anhydrous USP 99.5% in a ratio of 6 to 1 by volume. One dose consists of 1.40 ml of the ointment, containing 1.20 ml of 1.62% testosterone gel and 0.20 ml of glycerin anhydrous USP 99.5%. The testosterone present in one dose of the ointment in Recipe A is calculated to be approximately 16.5 mg, based on mass readings taken while following the recipe method and on information published in the labeling of 1.62% testosterone gel. Two doses are used daily, with one dose applied circa 8 am and the second dose applied circa 3 pm.

In Recipe B, the ointment consists of a mixture of 1.62% testosterone gel with glycerin anhydrous 99.5% in a ratio of 0.915 g of gel to 0.20 ml of glycerin. The quantity of ointment thus created is applied using ten square centimeters of ointment-bearing contact area of the device. Dosing need not be applied in exactly these quantities, but these ratios, of gel mass to glycerin volume and of gel mass to contact area, are used in all doses under this recipe.

With Recipe B, two doses are applied daily, but the doses may not be equal to each other. For Amneal® 1.62% testosterone gel, the dose applied circa 8 am consists of 1.098 g of 1.62% testosterone gel and 0.24 ml of glycerin anhydrous 99.5%, for a total mass of circa 1.4 g and a total volume of circa 1.54 ml. The dose applied circa 3 pm consists of 0.822 g of 1.62% testosterone gel and 0.18 ml of glycerin anhydrous 99.5%, for a total mass of circa 1.05 g and a total volume of circa 1.15 ml. The testosterone present in these doses of the ointment is calculated to be circa 17.8 mg in the 8 am dose and circa 13.3 mg in the 3 pm dose, based on mass readings taken while following the recipe method and on information published in the labeling of Amneal® 1.62% testosterone gel.

For Lupin® 1.62% testosterone gel, the dose applied circa 8 am consists of 1.006 g of 1.62% testosterone gel and 0.22 ml of glycerin anhydrous 99.5%, for a total mass of circa 1.283 g and a total volume of circa 1.4 ml. The dose applied circa 3 pm consists of 0.732 g of 1.62% testosterone gel and 0.16 ml of glycerin anhydrous 99.5%, for a total mass of circa 0.933 g and a total volume of circa 1.0 ml. The testosterone present in these doses of the ointment is calculated to be circa 16.3 mg in the 8 am dose and circa 11.9 mg in the 3 pm dose, based on mass readings taking while following the recipe method and on information published in the labeling of Lupin® 1.62% testosterone gel.

With either recipe, proper ointment preparation requires that the gel component and the glycerin component be fully intermixed prior to use. For small quantities, such as single doses of either recipe, allowing the two ointment components to stand in contact with one another in a tightly sealed container at room temperature for a period of 72 hours suffices.

The device consists of a minimum of three layers with an optional fourth layer. One layer consists of gauze having thickness of approximately 2 mm uncompressed, and having area of 12 square cm (Recipe A and 8 am doses of Amneal® Recipe B) or 9 square cm (3 pm doses of Amneal® Recipe B) or 11 square cm (8 am doses of Lupin® Recipe B) or 8 square cm (3 pm doses of Lupin® Recipe B), such as may be cut from 6-ply bandage gauze or 6-ply crinkle gauze. One layer consists of adhesive membrane which is medically appropriate for skin contact, sized so that when the gauze is centered within and adhered to the adhesive membrane, adhesive perimeter is present around the gauze sufficient to maintain attachment to skin for up to circa 40 hr. The third layer consists of metalized foil, such as that used in construction for ensuring airtight joints in ductwork for heating, ventilation, and air conditioning. The metalized foil layer is sized to match the membrane layer. The device also includes a sliver of paper. The optional layer consists of adhesive membrane sized at least 0.5 cm larger on all sides than the metalized foil layer.

### BRIEF DESCRIPTION OF DRAWINGS

FIG. 1 is a depiction of the device, showing top, front, and side views. In this FIGURE, the top view depicts the face of the device which is applied to the skin. Circled number 1 indicates the metalized foil layer. Circled number 2 indicates the adhesive membrane layer. Circled number 3 indicates the gauze layer. Circled number 4 indicates the sliver of paper.

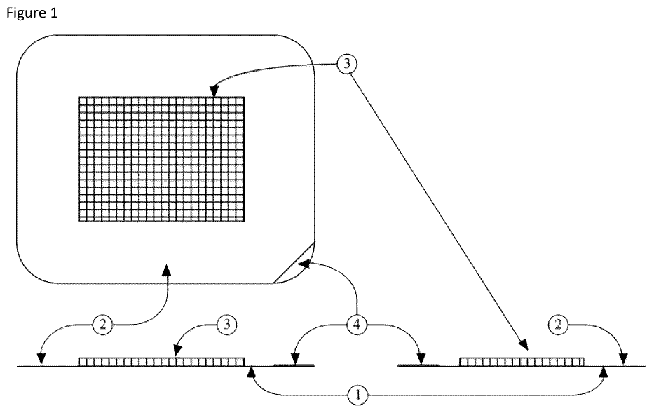

### DETAILED DESCRIPTION OF THE INVENTION

The applicant has discovered that 1.62% testosterone gel may be held in continuous contact with unbroken nonscrotal human skin for a period of up to 40 hours without material adverse consequence if the gel is first combined with USP glycerin 99.5% in a volume:volume ratio of between 6:1 and 4:1 to create an ointment. The applicant has devised practical methods of creating the ointment, using commonly available tools and supplies. The applicant has further created an effective and practical device for holding such ointment adjacent to skin, where the skin contact of the gel is confined to a specific predetermined surface area, the testosterone in the gel is protected against decomposition resulting from exposure to UV radiation such as is naturally present in sunlight, and all components of the ointment are physically confined so as to mitigate both the possibility and the perception of the possibility of accidental transfer of testosterone to a non-patient person or creature. The applicant has demonstrated that usage of the device and method results in therapeutically effective treatment of hypogonadism in a research subject, and that the therapeutic effectiveness exceeds that of 1.62% testosterone gel used in isolation per its FDA labeling on that research subject. The applicant has demonstrated that the usage of the device and method mitigates the perception of residual risk of accidental exposure to testosterone. The applicant has further demonstrated therapeutic efficacy using moderately less total administered testosterone than with AndroDerm®, and omitting ingredients included in prior art as documented in U.S. Pat. No. 5,152,997.

After combining 1.62% testosterone gel with glycerin anhydrous USP 99.5%, the ointment consists of ethanol, a carbomer (such as carbopol 980 or carbomer homopolymer type C), glycerin, isopropyl myristate, purified water, an alkalizing agent (such as sodium hydroxide), and testosterone. Calculations for ointment Recipe A indicate ethanol circa 61.9% volume/volume, glycerin circa 14.2% volume/volume, and testosterone circa 1.3% mass/mass or 1.18% mass/volume. Calculations for ointment Recipe B indicate ethanol circa 61.0% volume/volume, glycerin circa 15.5% volume/volume, and testosterone circa 1.27% mass/mass or 1.16% mass/volume.

For comparison, U.S. Pat. No. 5,152,997 documents an ointment having ethanol preferably about 50% by volume, and having glycerin preferably 20% to 30% by volume. The ointment herein documented is consistent with the greatest 24 hr cumulative flux range for testosterone concentration as documented in Table 1 of U.S. Pat. No. 5,152,997, but deviates from the prior art preferences in that patent for both ethanol percentage and glycerin percentage. Additionally, the prior art of U.S. Pat. No. 5,125,997 incorporates glycerol monooleate and methyl laurate in all disclosed formulations, while neither glycerol monooleate nor methyl laurate are present in recipes disclosed herein.

Even though the ointment percentages are on the high side for ethanol and the low side for glycerin, the usage experience of the research subject with the ointment, device, and method resulted in no vesicles (Recipe B) or very rare appearance of vesicles (one occurrence in six months with Recipe A) and only mild erythma. The research subject notes that the erythma resulting from usage of ointment, device, and method is no worse than erythma which resulted from usage of AndroDerm® according to its labeling, during the time period that the research subject used AndroDerm® regularly.

In contrast, usage of unmodified gel with a similar device and method resulted in marked erythma and occasional vesicles. Usage of unmodified gel with the device is therefore not advised and would be inconsistent with the methods here documented.

One method of creating one to several doses of Recipe B in a specific dosing quantity may be performed using the following ingredients, materials, and supplies: 1.62% testosterone gel supplied in a pump bottle from an FDA approved manufacturer (such as can be prescribed for the treatment of conditions of testosterone deficiency), glycerin anhydrous 99.5% USP, clean slip tip 3 ml syringes of a design for which the plunger may be removed from and subsequently reinserted in the barrel without substantial loss of integrity, caps for slip tip syringes, a mass balance or electronic scale with balance tray of a size adequate to hold a 3 ml syringe and of accuracy +/−0.001 gram and capacity at least 2 gram greater than the mass of one empty syringe, a 1 ml slip tip syringe marked in 0.02 ml increments, a block or wedge or watchglass or other small ledge on which a syringe-sized item may be placed such that its tip is in midair and not in contact with a work surface, a flat and level work surface of approximately 30 cm by 60 cm, disposable tissues and/or paper toweling as needed, materials for marking or labeling syringes, a metalized pouch or sleeve of a size which can contain several syringes with plungers partially extended, and such personal protection equipment as is considered appropriate for handling the ingredients without substantial risk of personal harm.

A method proceeds as follows. Calculate the desired mass of gel per dose, based on the labeled strength of 1.62% mass/mass; this equals a mass ratio of testosterone to gel of 16.2 mg of testosterone per 1.00 g of gel. The mass ratio is also expressible as 1.00 g of gel containing 16.2 mg of testosterone. As a sample calculation, for an intended dose amount of 11.9 mg of testosterone as externally applied, the calculated gel amount would be

$$11.9 mg \times ( 1. g / 1 6.2 mg ) = 0.7 \; 35 g$$

Having calculated the desired mass of gel for the intended dosing, compute the volume of glycerin using the Recipe B ratio of 0.915 g of gel to 0.20 ml of glycerin. To continue the sample calculation,

$$0.7 \; 35 g \times ( 0.2 ml / 0.9 \; 15 g ) = 0.161 ml$$

Depending on available measuring tools and their precision, the practitioner may opt to reverse the calculation order from a volume measure which is more suited to available equipment and close to the calculated glycerin volume. For example, when using a syringe which is marked in increments of 0.02 ml, the practitioner might reasonably think that a volume of 0.160 ml would be more precisely measured on a repeated basis than a volume of 0.161 ml. Reversing the order of calculation from 0.160 ml through the Recipe B ratio yields a gel mass of

$$0.1 \; 60 ml \times ( 0.915 g / 0 \text{.20} ml ) = 0.7 \; 32 g$$

For the gel mass of 0.732 g, the testosterone contained therein is

$$0.7 \; 32 g \times ( 16.2 mg / 1. g ) = 11.8 \; 58 mg$$

When rounded to the three significant figures available on the FDA labeling, these adjusted dosing quantities of 0.16 ml of glycerin and 0.732 g gel deliver an amount of testosterone indistinguishable from the desired 11.9 mg, in the Recipe B ratio.

Having calculated the desired target mass of gel and the desired volume of glycerin per dose, further prepare by donning personal protection equipment as appropriate. Extinguish any sources of flame near the work area, and remove sources of sparks or intense heat from the work area. To minimize the risk of accidental burns while following the method, do not light or smoke cigarettes, or introduce other sources of flame, spark, or intense heat during the process, as the gel contains a substantial proportion of flammable alcohol.

A method continues as follows. Remove the pump bottle from its packaging, and remove the cap from the pump bottle. If the pump has not been previously used, prime the pump per packaging directions.

A method continues as follows. Label one syringe as a holding syringe. Label one syringe as a dose-bearing syringe. Remove the plunger from the dose-bearing syringe and place on the block. Dispense a small amount of gel from the pump bottle into the barrel of the dose-bearing syringe. Reinsert the plunger into the dose-bearing syringe. Remove the plunger from the holding syringe and place on the block. Dispense gel from the dose-bearing syringe into the barrel of the holding syringe until the plunger is fully depressed and the tip of the dose-bearing syringe is filled with gel. Reinsert the plunger into the barrel of the holding syringe and depress the plunger in the holding syringe until most of the air is expelled from the barrel, but the gel remains in the barrel of the holding syringe. Place the dose-bearing syringe on the scale and tare the scale.

A method continues as follows. Remove the dose-bearing syringe from the scale, remove its plunger, and dispense one full pump of gel from the pump bottle into the barrel of the dose-bearing syringe. Depending on technique, it may be necessary to pause mid-pump and, holding the barrel vertically with tip downward, tap the syringe on the work surface; this moves the relatively viscous gel farther down the barrel and allows room for completing the pump depression. Pausing to tap as needed, complete dispensing one full pump of gel into the barrel of the dose-bearing syringe. Replace the plunger in the dose-bearing syringe and expel air to the extent practical while retaining all gel in the barrel. Place the syringe on the scale and observe mass. For a just-primed or recently-used pump, a single-pump mass of 1.15 g to 1.27 g is typical. For a previously-primed pump on its first use of the day, a single-pump mass of 1.05 g is not uncommon.

A method continues as follows. Remove the plunger from the holding syringe and place on the block. Dispense gel from the dosing syringe into the barrel of the holding syringe, and re-check the net gel mass of the dosing syringe on the scale. Iterate dispensing and re-checking as appropriate to reduce the net gel mass in the dosing syringe to within an acceptable tolerance of the target mass calculated for the dose. If too much gel is dispensed, such that the net mass of gel in the dosing syringe drops below the target mass by greater than the allowable tolerance, gel can be reintroduced to the barrel of the dosing syringe from the holding syringe after first reinserting the plunger into the holding syringe and removing the plunger from the dosing syringe. After reintroduction of more gel into the dosing syringe from the holding syringe, reinsert the plunger in the dosing syringe, remove the plunger from the holding syringe, and resume iteration of reducing net gel mass in the dosing syringe toward target mass. With practice and careful attention, the practitioner of this technique is able to achieve a tolerance of net gel mass in the dosing syringe at +/−0.010 g (+/−10 mg) of the target mass.

A method continues as follows. When the net mass of gel in the dosing syringe is within acceptable tolerance of the target mass for the dose, reinsert the plunger into the holding syringe, expel most air from the holding syringe while retaining all gel in the barrel, and set aside the holding syringe. Remove the cap from the glycerin container. Using the 1 ml syringe, draw up the target volume of glycerin as calculated for the dose. Remove the plunger from the barrel of the dosing syringe and place the plunger on the block. Holding the barrel of the dosing syringe close to horizontal, with the tip slightly lower, dispense the measured volume of glycerin from the 1 ml syringe into the barrel of the dosing syringe. Reinsert the plunger of the dosing syringe, taking care to keep the dosing syringe close to horizontal until the plunger is seated within the barrel, and then raising the tip above horizontal while expelling air. During the process of introducing glycerin to the dosing syringe, if the tip of the syringe drops well below horizontal long enough for glycerin to flow out of the tip, the dose is unrecoverable and must be scrapped.

A method continues as follows. Expel most air from the dosing syringe while retaining all fluids within the syringe. Apply a slip tip cap to the tip of the syringe, and cap tightly. Place the dosing syringe in a metalized sleeve or metalized pouch.

Multiple successive doses may be prepared, one at a time, by resuming the above method at the instruction of labeling a syringe as a dose-bearing syringe. When preparing multiple successive doses in succession, accumulated gel in the holding syringe may be used from time to time in place of a full pump of of gel from the pump bottle. When the desired quantity of doses of a given calculation have been prepared, set aside in the metalized sleeve or metalized pouch for at least three days (72 hours) in a dark place at room temperature. If testosterone gel remains in the holding syringe at the conclusion of a preparation session, dispose of such gel per pump labeling directions for disposing of gel expelled during pump priming.

At time of use of a dosing syringe, remove from metalized sleeve or metalized pouch. Uncap the syringe and slowly draw the plunger back. If schlieren (streaks) are observed within the fluid within the barrel of the syringe as the plunger is drawn back, the fluids of the gel and glycerin have not yet fully intermixed into one homogeneous ointment. In this circumstance, re-expel air from the syringe, recap, return the syringe to the metalized sleeve or pouch, and select another.

The method of above documented is not the sole method of creating the ointment. The method above documented is an example of a practical method which may be successfully performed by an individual who has a legitimate prescription for 1.62% testosterone gel but who is not trained in pharmaceutical compounding, making use of commonly available supplies and employing techniques which, while requiring ordinary care, do not require specialized training or skill. Other methods of creating the same ointment or an equivalent ointment are also envisioned, such as methods employing the tools, supplies, raw materials, and skills utilized by one skilled in the art of pharmaceutical compounding, or methods using industrialized control to obtain the appropriate ratio of ingredients comprising the ointment.

The device is constructed of multiple components. One of the components is metalized foil. One of the components is adhesive membrane. One of the components is gauze. One of the components is paper.

The metalized foil is selected for having the properties of being practically impervious to gases, vapours, and liquids at ordinary temperatures and pressures, of being supplied with or compatible with an adequately strong contact adhesive, of being opaque to visible and ultraviolet light, and of being reasonably malleable in that the foil will conform to the contours of the surface to which it is applied. The specific foil used in construction of device prototypes is Nashua 322 Multi-Purpose Foil Tape. This metalized foil tape is advertised for general metal repairs and marketed for use in the heating, ventilation, and air conditioning (HVAC) industries. This tape is readily available on rolls of 72 mm width and 46 meters in unrolled length.

The adhesive membrane is selected for having the properties of having a skin-compatible contact adhesive, of being supplied in a width exceeding that of the metalized foil, of having a facing paper which can be marked with an ink pen, and of being substantially impervious to the adhesive used on or with the metalized foil. The specific adhesive membrane used in construction of device prototypes is Smith & Nephew Opsite Flexifix. This adhesive membrane is readily available on rolls of 100 mm width and 10 meters in unrolled length.

The gauze is selected for having the properties of being machinable into a rectangle or other shape of known area, of keeping to its machined shape reasonably closely under moderate manual handling such as occurs during device assembly, of being safe to use in extended contact with unbroken skin, of being constructed of material to which the ointment will cling under moderate manual handling such as occurs during device assembly and dose application, of being constructed of material which adheres to the adhesive membrane, of having adequate depth such that the open space within its meshwork is sufficient to contain the volume of applied gel appropriate to a specific surface area, and of containing sufficient open space within its meshwork such that compounds dissolved in a gel or ointment impregnated into the gauze may move freely. Multiple specific gauzes have been used in construction of device prototypes, including Med Pride Krinkle Gauze Roll 6 Ply MPR-60376. Med Pride Krinkle Gauze Roll is available in prepacked rolls of nominal size 4.5 inch width and 4.1 yards in unrolled length.

The paper is selected for having the properties of being easily machinable, readily available, adhering to the adhesive membrane, and not itself being adhesive. The paper component is a byproduct of device prototypes being constructed of other premade components of which the adhesive covers the entire skin-facing surface. The paper covers a small area of the otherwise skin-adhering surface of the device, to form a tab which does not adhere to the skin. The tab facilitates eventual device removal. If a comparable device were constructed via a device-specific process involving device-specific deposition of adhesive, the adhesive could be omitted from the tab portion of the device. This approach would also facilitate device removal from the skin without requiring the use of paper in forming a tab. Such alternate construction is considered construction of an equivalent device. Thus, the paper component is not integral to the nature of the device but is instead an artifact of the specific materials and methods of construction of prototype devices.

Device pre-assembly consists first of adhering the metalized foil to the medically appropriate adhesive membrane such that the adhesive on or used with the foil is covered by the non-adhesive face of the membrane. So doing may involve removing a facing paper from the adhesive face of the foil tape, or removing a protective layer from the non-adhesive face of the adhesive membrane, or both. This step of the pre-assembly method creates a two-layer lamination of the metalized foil and the medically appropriate adhesive membrane, with the facing paper of the adhesive face of the adhesive membrane still in place and undisturbed.

Device pre-assembly continues by trimming the lamination to a rectangle of face size of 61+/−1 mm by 71+/−1 mm, and rounding each of the four corners of the rectangle to a 10 mm radius. Device pre-assembly continues by using an ink pen to mark the facing paper of the medically appropriate adhesive membrane with a line at a distance of 15+/−1 mm from the seam in the facing paper, or from one of the bounding edges of the facing paper. Device pre-assembly continues by machining the gauze into rectangles of 30+/−1 mm by 40+/−1 mm, 27.5+/−1 mm by 40+/−1 mm, 20 mm+/−1 mm by 40+/−1 mm, or squares of 30+/−1 mm by 30+/−1 mm, or other suitable shape not exceeding 31 mm by 41 mm, depending on the surface area required for the applicable recipe and dosage. Device pre-assembly continues by machining the paper into a shape bounded on one part of its edge by a quarter circle of 10 mm radius, by continuing the tangents at each end of the quarter circle by a distance in the range of 0 mm to 5 mm, and on the remainder of its edge by a straight line connecting the points on either end of the tangents. Device pre-assembly of the optional layer continues by cutting a rectangle of the medically appropriate adhesive membrane of face size 80+/−1 mm by at least 90 mm, up to 100 mm, and rounding each of the four corners of the rectangle to a 10 mm radius.

Following device pre-assembly, the described results or end products of pre-assembly may be stored indefinitely until the time of dose administration. At the time of dose administration, the pre-assembled device materials used with administration of one dose consist of one laminated rectangle, one precut gauze of a face area appropriate to the dose being administered, one optional layer if applicable to circumstances, and one to two paper slivers depending on whether the optional layer is used.

Device assembly consists of first removing one part of the facing paper from the adhesive face of the adhesive membrane, and attaching the paper sliver to one uncovered corner of the adhesive. The sliver of paper is attached to a corner of the membrane such that its quarter circle edge aligns with the rounded corner of the lamination at the perimeter of the lamination. Once the paper sliver is in place, reapply the removed facing paper to the adhesive, covering the sliver of paper, with the slick side of the facing paper against the adhesive; this is the same as the orientation when packaged. In the reapplication of the facing paper to the adhesive, apply the facing paper square to the laminated rectangle, but obscure only approx 1 cm of the adhesive inward from the short edge of the lamination, leaving the center area of the adhesive exposed. A preapplied ink pen mark on the facing paper may be used as a guide in this step.

Continue device assembly by removing the second part of the facing paper from the adhesive face of the lamination. The reapplied first piece of facing paper may be used to stabilize the lamination without adversely affecting the tack of the adhesive. Reapply the second piece of facing paper to the adhesive, square to the lamination, obscuring 1.5 cm of the adhesive from the short edge of the lamination. A preapplied ink pen mark on the facing paper may be used as a guide in this step.

Continue device assembly by applying the gauze to the adhesive of the laminated membrane, inset at least 1.2 cm on all edges. The 1.5 cm overlap of the reapplied second part of the facing paper creates an edge to which the gauze may be set during placement of the gauze on the adhesive. When using a rectangular size of the gauze, the orientation of the gauze on the membrane is square to the membrane and such that the shorter, 20 to 30 mm dimension of the gauze is centered on the 61 mm dimension of the membrane and the 40 mm dimension of the gauze is centered on the 71 mm dimension of the membrane. When using the square size of the gauze, the orientation of the gauze is square to the membrane, centered in the short dimension, and approximately centered in the long dimension. The sizes of the gauze and lamination are such that at least approximately 15 mm of adhesive will bound the gauze on all four sides, when the gauze is accurately placed. The ink pen mark on the facing paper may be used as a spacing aid during hand assembly.

Immediately subsequent to assembly, the method of usage consists of applying one dose of ointment to the center of the gauze. The adhesive face of the anointed device is then applied to clean, dry, bare, intact non-scrotum skin in a location not over a bony prominence and free of excessive skin flexion. Suitable skin areas include the inner and outer thigh, and the upper arm. In the application of the device to the skin, first remove the facing paper which covers the paper sliver, and handle the anointed device using the second facing paper. Apply the adhesive edge surrounding the paper sliver as the first point of contact to skin, flexing or cupping the membrane during application to bring the adhesive face into contact with the skin without compressing the gauze into the skin. After enough adhesive face is in contact with skin to fix the positioning of the device, remove the remaining facing paper and adhere the adhesive to skin around the full perimeter. Trace over the perimeter of the attached device with a finger to ensure adhesion before pressing the center of the device toward the skin.

If extended submersion in water is anticipated, or if excessive sweating is anticipated, or in any other circumstance in which there may be a possibility or the perception of the possibility of unintended or accidental device removal, the optional adhesive membrane layer may be applied over the top of the main device, squared to and centered over the main device, with an additional sliver of paper applied to one corner of the optional layer. This layer aids in securing of the main device to the skin, mitigating the possibility and the perception of the possibility of unintended or accidental removal.

After application, the device is left in place for circa 30 to 40 hours. To remove, peel away from skin, beginning at the location at which the paper sliver prevents adhesion of the membrane to skin. On removal, dispose of the removed device away from children and pets. Wash the skin area which was under the device, and any residue, with soap and warm water.

The research subject administers two devices daily using one ointment dose per device, applying one dosed device at circa 8 am to 9 am and the second at circa 3 μm to 4 pm, with both devices retained until circa 11 pm the following evening, for an application duration of circa 38 hours and 32 hours for the morning and afternoon applications respectively. The research subject reports that his quality of life under this protocol is substantially similar in many respects to that realized through his prior use of the since-discontinued AndroDerm® patches. The research subject reports that from an inventory management standpoint, the ointment Recipe A protocol is somewhat simpler than the ointment Recipe B protocol. The research subject further reports that under either protocol, caffeine intake returned to individually typical levels, work productivity is good, mood is manageable, energy level is good, and libido is good including after showering.

The research subject reported that in use of devices, no liquids or fluids escape through the metalized foil of the device. When using the device without the optional additional adhesive membrane layer, very occasionally fluid seeped from an edge of the secured metalized foil, consistent with spot failure of adhesive along a channel from the gauze to the device edge; when occurring, this issue was entirely attributable to significant sweating, significant skin flexion, or significant water exposure such as during extended bathing or swimming. Routine and regular application of the optional additional membrane layer, covering and surrounding the metalized foil area of the device, has fully prevented recurrence of fluid seep including when sweating, when the device is on an area of skin flexion, or during bathing or swimming. The research subject additionally notes that while using devices and independently of specific clothing choices, interpersonal physical contact including intimate contact is not inhibited by the perception of any risk or residual risk of accidental transfer of testosterone to others.

It may be noted that the research subject's dose of AndroDerm® patches was two of the nominal 4 mg size daily. Each such “4 mg” AndroDerm® patch contained 19.5 mg of testosterone, for a total application of 39 mg of testosterone daily on the AndroDerm® regimen. On the regimen using the device and ointment Recipe A described herein, the research subject applied 33 mg of testosterone daily. On the regimen using the device and ointment Recipe B, the research subject applied 31.1 mg of testosterone daily with Amneal® and 28.2 mg of testosterone daily with Lupin®.

| Protocol | Daily Applied T (mg) | Date | T (ng/dl) |
|---|---|---|---|
| AndroDerm ® 4 mg am + AndroDerm ® 4 mg pm | 39.0 | Aug. 25, 2021 | 945 |
| AndroDerm ® 4 mg am + AndroDerm ® 4 mg pm | 39.0 | Dec. 27, 2022 | 703 |
| 1.62% testosterone gel, 4 pumps am | 81 | May 4, 2023 | 104 |
| Amneal ® Recipe A, 1.4 ml am + 1.4 ml pm | 33.0 | Aug. 3, 2023 | 836 |
| Amneal ® Recipe A, 1.4 ml am + 1.4 ml pm | 33.0 | Nov. 6, 2023 | 789 |
| Amneal ® Recipe B, 1.40 g am + 1.05 g pm | 31.1 | Mar. 12, 2024 | 794 |
| Lupin ® Recipe B, 1.28 g am + 0.93 g pm | 28.2 | Jul. 24, 2024 | 603 |

As compared to the AndroDerm® regimen, the protocol of applying four pumps of 1.62% testosterone gel according to directions in product labeling failed to address the subject's testosterone deficiency. The ointment Recipe A protocol and the ointment Recipe B protocol each addressed the subject's testosterone deficiency to a degree comparable to the AndroDerm®, when evaluated on the basis of a testosterone level determined from a morning peripheral blood sample and also on the basis of the subject's experience of quality of life.

The use of metalized tape as the external layer of the device, fully covering the ointment application area and with full perimeter adhesion to the skin, fully separates the ointment from the external environment and thus prevents the irradiation of the ointment by sunlight. This in turn prevents the photodegradation of testosterone as otherwise occurs when testosterone in aqueous solution is irradiated under ultraviolet (UV) light in spectra present in sunlight (Meite et al, 2016.) This is in contrast to an approach which relies on clothing to cover the testosterone application area. Depending on the material of its construction, clothing may not be fully opaque to UV light. Also, clothing may shift, slide, or ride up, or in warm weather clothing may be deliberately removed. Any such occurrences could expose gel applied per FDA labeling to UV light, leading to photodegradation of the testosterone present in the gel or on the skin. The full perimeter adhesion of the metalized foil tape thus mitigates the risk of photodegradation of testosterone during use by the subject, and does so more effectively than can be expected of a clothing-based approach.

The use of metalized tape as the external visible layer of the device contributes to the mitigation of the perception that there exists any residual risk of accidental transfer of testosterone. Its presence as the visible outer layer of an adhesive device contributes to the perception that the device fully confines the ointment on the subject's skin, as opposed to porous, non-adhered clothing. Also, the full perimeter adhesion of the device contributes to the mitigation of the risk and of the perception of residual risk of transfer, in that a fully adhered device will not slide, shift, ride up, or otherwise become easily displaced in the manner that clothing may. Additionally, clothing which is worn over an area of applied gel per FDA labeling may lead to accidental transfer of testosterone if handled by others prior to laundering (Miller, Rogol, and ZumBrunnen, 2012). In contrast, the used device is disposed of in trash, and any residual testosterone on the device goes into the trash with the device. The immediate disposal of the removed device at end of use further mitigates the residual risk and the perception of residual risk of accidental transfer of testosterone.

The device herein described deviates from the prior art of U.S. Pat. No. 5,152,997 in that this device specifies a metalized foil layer for the specific purposes of the physical containment of all ointment components, and mitigation of the risk of photodegradation of testosterone via irradiation by UV light, for the duration of use. The device herein described further deviates from the prior art of U.S. Pat. No. 5,152,997 in the composition of the ointment. The device herein described further deviates from the prior art of U.S. Pat. No. 5,152,997 in that a five-layer patch is not required for safe and effective use of the ointment. The device herein described further deviates from the prior art of U.S. Pat. No. 5,152,997 in that the device herein described may be constructed from readily available materials using readily available low-cost tools.

The device and methods herein described, in particular the Recipe B ointment and related methods, deviate from and improve on prior patents and most previously available therapies by practical availability of fine-grained patient-specific dosing adjustment. Of known FDA approved therapies, only intramuscular injections of testosterone esters provide a comparable granularity of dosing adjustment.

As compared to intramuscular injections, the device and methods herein described provide comparable granularity of dosing adjustment without injection site pain, without buildup of scar tissue at injection sites, with shorter intervals between dosing adjustments and subsequent laboratory tests, and with better consistency from one day to the next of the circulating peripheral testosterone level and of the patient experience of quality of life.

1. A device comprising:
a) gauze or comparable material which a gel or ointment will adhere to, permeate, and move freely within;
b) adhesive membrane appropriate for use on unbroken nonscrotal human skin for durations of up to 40 hours; and
c) metalized foil
for which the gauze is centered on the adhesive face of the adhesive membrane, the face area of the gauze is between four (4) square centimeters and fifteen (15) square centimeters, the adhesive membrane extends beyond the gauze to at least a degree appropriate to ensure the adhesion of the device to the skin for a duration of up to 40 hours, and the metalized foil is adhered to the opposite surface of the adhesive membrane from the gauze and fully covers the surface of the adhesive membrane opposite the gauze and also fully covers the surface of the adhesive membrane for an additional margin around the gauze not to exceed that of the adhesive membrane.

2. The device of claim 1 to which a thin layer of nonadhesive material is applied to a small area of the adhesive face abutting the perimeter of the adhesive membrane, or in which a small area abutting the perimeter of the face which is otherwise generally adhesive is rendered non-adhesive by other means, so as to form a nonadhesive tab to facilitate removal of the device, without affecting so large an area as to compromise the intended adhesion of the device during intended use.

3. An ointment comprising
a) 1.62% testosterone gel, itself comprised of ethanol, carbomer, isopropyl myristate, sodium hydroxide, purified water, and testosterone, as approved by the United States Food and Drug Administration; and
b) glycerin anhydrous 99.5% USP
for which the volume:volume ratio of testosterone gel to glycerin anhydrous USP 99.5% is between six to one (6:1) and four to one (4:1) inclusive.

4. A method of providing testosterone therapy to a human comprising determining the magnitude of dose of an ointment comprising ethanol, carbomer, isopropyl myristate, sodium hydroxide, purified water, testosterone, and glycerin anhydrous 99.5% USP, including such ointment for which the ingredients other than glycerin are supplied as 1.62% testosterone gel as approved by the United States Food and Drug Administration, production of a dose of said ointment, sizing of a device comprising gauze, adhesive membrane appropriate for use on unbroken nonscrotal human skin, and metalized foil such that the gauze face area is sized appropriately to the magnitude of the dose of the ointment, constructing and assembling said device, placing said dose onto the gauze of said device, and adhering said dosed device to an area of the unbroken nonscrotal skin of the human.

5. The method of claim 4 within which each device adhered to the skin remains adhered for a total time materially exceeding 24 hours and not exceeding 40 hours.

6. The method of claim 5 within which more than one dosed device may be adhered to the skin of the same human on the same day, on different skin areas, at materially different times of day, with the doses used within one day being of equal magnitude to one another or of different magnitudes from one another.
